(19) United States
(12) Patent Application Publication
(10) Pub. No.: US 2013/0123603 A1
(43) Pub. Date: May 16, 2013

(54) MEDICAL DEVICE AND METHOD FOR DISPLAYING MEDICAL IMAGE USING THE SAME

(71) Applicant: SAMSUNG ELECTRONICS CO., LTD., Suwon-si (KR)

(72) Inventor: Seung Woo SHIN, Yongin-si (KR)

(73) Assignee: SAMSUNG ELECTRONICS CO., LTD., Suwon-si (KR)

(21) Appl. No.: 13/673,335

(22) Filed: Nov. 9, 2012

(30) Foreign Application Priority Data
Nov. 15, 2011 (KR) 10-2011-0118911

(51) Int. Cl. A61B 6/00

(52) U.S. Cl. CPC A61B 6/463; A61B 6/566; A61B 6/5217; A61B 6/465; A61B 6/467; A61B 6/563; A61B 6/466
USPC 600/407

(57) ABSTRACT

Disclosed is a medical device which displays an object relating to an image considered to include an indication relating to a pathological entity, such as, for example, a lesion, on a terminal on which a medical image is displayed, and a method for displaying a medical image by using the same. The medical device includes a display device, a communication device which communicates with an external device, and a controller which displays an image which includes the indication relating to a pathological entity on the display device when the communication device receives image information relating to a subject.

### CROSS-REFERENCE TO RELATED APPLICATION

This application claims priority from Korean Patent Application No. 10-2011-0118911, filed on Nov. 15, 2011, in the Korean Intellectual Property Office, the disclosure of which is incorporated herein by reference in its entirety.

### BACKGROUND

1. Field

Exemplary embodiments relate to a medical device. In particular, exemplary embodiments relate to a medical device and a method for displaying a medical image by using the same, in order to reduce medical image interpretation time required by medical workers and thereby provide efficient diagnosis.

2. Description of the Related Art

In general, clinical diagnosis has an important role in treatment of patients in medical operations, development of medical techniques greatly contributes to accurate clinical diagnosis, and dependence thereon will continue to increase.

Accordingly, although medical imaging devices, such as computer tomography (CT), magnetic resonance imaging (MRI) and X-ray devices have become essential in modern medicine, medical services are often accomplished as a result of an inefficient process requiring a long time and great labor, in which abnormal sites of patients are imaged with medical imaging devices, the images are printed onto films, the films are transferred to an attending physician of the patients, and the attending physician finally performs clinical diagnosis. As a consequence of this inefficiency, physicians have a reduced capacity for providing rapid and accurate treatment.

However, in accordance with development of computer and communications technology, systems for providing medical services by using computer and data communications technology are currently being researched and developed in medicine. For example, a medical imaging system (e.g., a picture archiving communication system: PACS) wherein a computer communication network is installed throughout the hospital, all X-ray films are converted into digital data, the digital data is entered into a database and stored in a large storage medium which is connected to a server, and an X-ray image of the target patient is checked at respective medical offices via a computer monitor has been introduced.

The medical imaging system (PACS) refers to a general digital image management and transfer system that acquires medical images, in particular, radiological diagnostic images, in digital form, transfers the images via a high-speed communication network, and diagnoses patients by using an image search device.

The medical imaging system (PACS) ultimately aims at constructing a filmless hospital system. For this purpose, it is necessary that technologies such as image display and processing, data communication and networking, database management, information management, user interfaces, data storage and management be generally constructed.

Respective devices constituting the PACS transfer and manage medical images by using a digital imaging and communications in medicine (DICOM) protocol.

DICOM refers to a communications protocol that effectively services communication between CT and MRI devices, as well as communication between various digital image acquisition devices, such as nuclear medicine and ultrasound devices, and other information (data) systems by using an industrial standard network.

In conventional technologies, expensive gateways are required in order to communicate data, or communication is impossible, because consistent standards are not necessarily used and methods for storing and communicating data may vary according to manufacturing companies, or based on the type and the model of imaging devices being used.

However, as DICOM standards become settled, devices which comply with these standards enable data communication, regardless of manufacturing companies and device types, without using specific converters. Such data communication is not limited to communication between DICOM service devices present in the hospital, and communication with remote sites is also possible.

Further, network construction has increasingly complied with standards widely used in the computer industry in recent years, and DICOM standards are thus readily applicable to communication relating to all medical image-associated systems, including communications between sites in the hospital, communications between remote medical offices, and remote diagnosis systems.

As such, the DICOM—based medical imaging system provides an infrastructure which is useful for reducing patient diagnosis time required by medical workers and for realizing an efficient diagnosis.

Further, in accordance with recent technological development, medical images may be three-dimensionally constructed and provided. Three-dimensional medical images may improve accuracy of lesion detection, but also may entail a longer time for interpretation of medical images and for detection of lesions, due to an increased amount of data which is provided in such images.

When a doctor who performs an initial medical examination interprets one or more three-dimensional medical images and finds one or more lesions, this lesion information is input into a medical imaging system and thus made available for other doctors to read this information. The other doctors may check diagnosis results stored by the first doctor, but may inefficiently be required to interpret the three-dimensional medical images again in order to check the corresponding lesions from images.

### SUMMARY

Therefore, in one aspect, exemplary embodiments provide a medical device which displays an object relating to an image considered to include an indication relating to a pathological entity, such as, for example, a lesion, on a terminal on which a medical image is displayed, and a method for displaying a medical image by using the medical device.

Additional aspects of the exemplary embodiments will be set forth in part in the description which follows and, in part, will be obvious from the description, or may be learned by practice of the exemplary embodiments.

In accordance with one aspect of one or more exemplary embodiments, provided is a medical device including: a display device; a communication device which communicates with an external device; and a controller which displays an image which includes an indication relating to a pathological entity on the display device when the communication device receives image information relating to a subject.

The controller may display an object relating to the image which includes the indication relating to the pathological entity on the display device.

The object may include at least one of a thumbnail and a shortcut of the image which includes the indication relating to the pathological entity.

The controller may display the image which includes the indication relating to the pathological entity in an enlarged form on the display device when a command relating to the object is received.

The display device may include a touch screen, and the command relating to the object may be received via a touch of the object displayed on the display device.

The medical device may include an input device which includes at least one of a remote controller, a mouse, a keyboard, a voice recognition device and a movement recognition device, and the command relating to the object may be received via the input device.

The image which includes the indication relating to the pathological entity may include an image which includes image information relating to a lesion.

The communication device may communicate with at least one of a server, the internet and a second medical device in order to receive the image information relating to the subject.

In accordance with another aspect, one or more exemplary embodiments provide a method for displaying a medical image including: receiving a plurality of images relating to a subject; and displaying a first image which includes an indication relating to a pathological entity from among the plurality of images relating to the subject on a medical display device, when the plurality of images relating to the subject are received.

The displaying the first image may include: displaying an object which relates to the first image on the medical display device; and displaying the first image in an enlarged form on the medical display device when a command relating to the object is received.

The command relating to the object may be received via a touch of the object displayed on the medical display device.

The command relating to the object may be received via at least one of a remote controller, a mouse, a keyboard, a voice recognition device and a movement recognition device.

The object may include at least one of a thumbnail and a shortcut of the first image.

The displaying the first image may include: displaying images relating to the subject on the medical display device and displaying at least one record relating to the first image on the medical display device.

The receiving the plurality of images relating to the subject may include receiving the plurality of images relating to the subject from at least one of a server, the internet and a medical device.

In accordance with another aspect, one or more exemplary embodiments provide a medical imaging system including: an image acquisition device which acquires a plurality of images relating to a subject; and a medical display device which displays a first image which includes an indication relating to a pathological entity from among the plurality of images relating to the subject.

The medical display device may display an object which relates to the first image.

The object may include at least one of a thumbnail and a shortcut of the first image.

The medical display device may display the first image in an enlarged form on the medical display device, when a command relating to the object is received.

The command relating to the object may be received via a touch of the object displayed on the medical display device.

The command relating to the object may be received via at least one of a remote controller, a mouse, a keyboard, a voice recognition device and a movement recognition device.

The medical display device may include at least one of a computer, a smart phone and a tablet personal computer (PC).

The medical display device may display at least one record relating to the first image.

The first image may include an image which includes image information relating to a lesion.

The medical imaging system may further include: a server which stores the acquired plurality of images relating to the subject and which transfers the stored plurality of images to the medical display device, wherein the server supports a digital imaging and communication in medicine (DICOM) protocol.

The image acquisition device may acquire three-dimensional images relating to the subject.

Each of the image acquisition device and the medical display device may support a DICOM protocol.

According to an aspect, medical workers may immediately see images which include lesions without causing an inefficiency in which medical workers redundantly interpret the images in order to find lesions.

### BRIEF DESCRIPTION OF THE DRAWINGS

These and/or other aspects of the present disclosure will become apparent and more readily appreciated from the following description of the exemplary embodiments, taken in conjunction with the accompanying drawings of which:

FIGS. 1 and 2 are views which illustrate a configuration of a medical imaging system according to an exemplary embodiment;

FIG. 3 is a view which illustrates a frame of the DICOM protocol format;

FIG. 4 is a view which illustrates a configuration of a terminal according to an exemplary embodiment;

FIGS. 5, 6, and 7 are views which illustrate respective states in which medical images are displayed on a terminal of a medical imaging system according to an exemplary embodiment;

FIGS. 8 and 9 are views which illustrate respective states in which images are displayed in enlarged forms on the terminal of the medical imaging system according to an exemplary embodiment; and

FIG. 10 is a flowchart which illustrates a method for displaying a medical image according to an exemplary embodiment.

### DETAILED DESCRIPTION

Hereinafter, exemplary embodiments will be described with reference to the accompanying drawings.

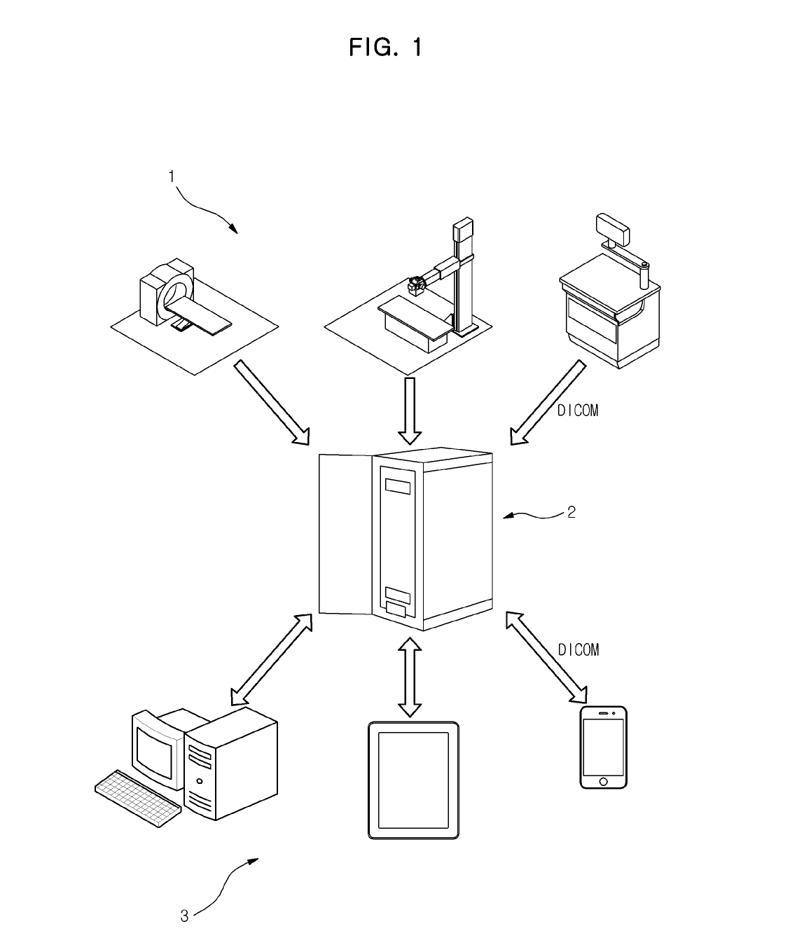

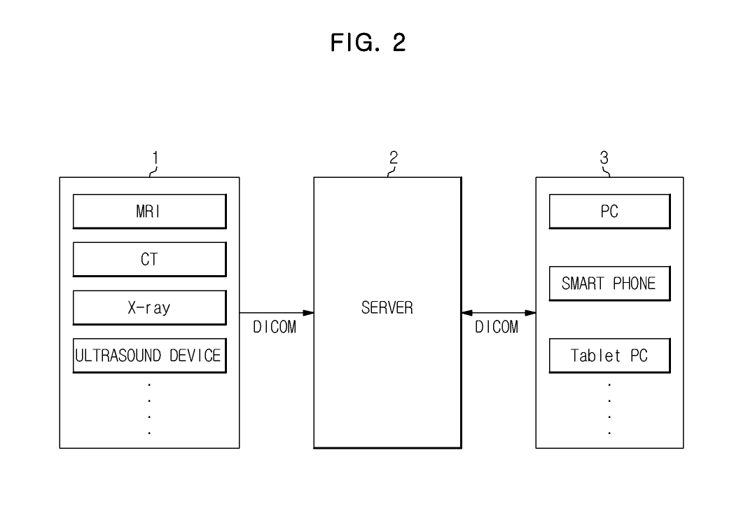

FIGS. 1 and 2 are views which illustrate a configuration of a medical imaging system according to an exemplary embodiment.

As shown in FIG. 1, the medical imaging system includes an image acquisition device 1 which acquires an image relating to a subject, a server 2 which receives the acquired image from the image acquisition device 1 and stores the received image in a medical display device 3, and the medical display device 3 which receives the image relating to the subject from the server 2 and displays the image.

As shown in FIG. 2, the image acquisition device 1 acquires three-dimensional images relating to subjects to be imaged, and may include, for example, a MRI device, a CT device, an X-ray device, and/or an ultrasound device or the like.

The image acquisition device 1 transfers three-dimensional images relating to the subject which are acquired by executing functions of respective devices to the server 2.

Subject image data which is transferred in the medical imaging system, including subject three-dimensional image data transferred from the image acquisition device 1 to the server 2, complies with a DICOM protocol format.

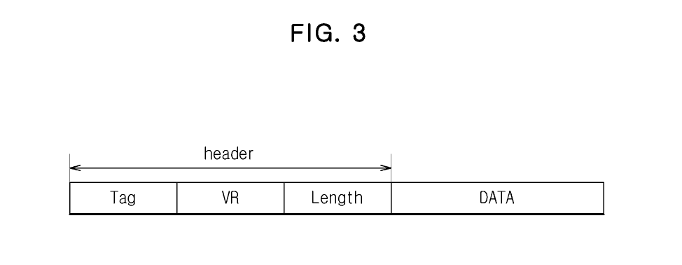

FIG. 3 is a view which illustrates a frame of the DICOM protocol format.

DICOM is an application network protocol that facilitates transfers of medical images, waveforms and other ancillary information and uses TCP/IP as an underlying transfer protocol.

A DICOM file includes a header and a data region. Various types of information which are required for DICOM file format, such as, for example, patient information, examination type, used apparatus, data size, diagnosis, resolution, number of color bits, play speed of images, communication information and encoding information, are stored in the header, and images and/or image data are stored in the data region.

The DICOM image file includes a single frame when the number of images stored in the data region is equal to one, and the DICOM image file includes a plurality of frames when the number of images is greater than or equal to two.

The server 2 stores three-dimensional image data relating to the subject, which has been transferred from the image acquisition device 1. The server 2 classifies transferred images based on predetermined classification criteria and stores the same.

Further, the server 2 transmits three-dimensional image data relating to the subject to the corresponding medical display device 3 based on a request received from the medical display device 3 in order to facilitate interpretation of the medical images. Transmission of data complies with the DICOM protocol format.

The medical display device 3 includes a terminal which displays one or more three-dimensional images relating to the subject, which images have been acquired by the image acquisition device 1, and the medical display device 3 receives one or more diagnosis records which relate to the acquired images. Generally, the diagnosis records have been created by medical workers, and the medical workers may perform a diagnosis by using the image displayed by the medical display device 3.

The medical display device 3 may include, for example, a personal computer (PC), or a portable smart phone, or a tablet PC. Any medical device is included within the scope of the present disclosure, provided that the medical device includes a program for displaying a file complying with a DICOM format and includes a display device which displays images.

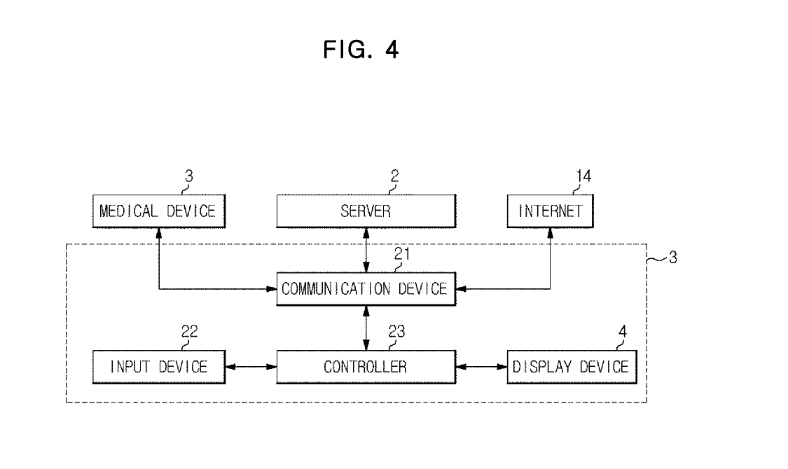

As described above, the medical display device receives three-dimensional image data relating to a subject via the server, but, referring also to FIG. 4, the medical display device 3 may receive medical images from a server of the medical imaging system and/or from an internet 14 or other computer or another medical device 3, and display the medical images.

FIG. 4 is a view which illustrates a configuration of the medical display device 3 according to an exemplary embodiment.

The medical display device 3 includes a communication device 21 that communicates with a server, an input device 22 which provides an interface for user operation of the medical display device 3, a controller 23 which controls the medical display device 3 based on commands received via the input device 22, and a display device 4 which displays medical images and diagnosis records and the like.

The communication device 21 receives images relating to subjects, and/or various information associated with a diagnosis performed by one or more medical workers, from the server 2, and transfers information which is modified or manipulated by the medical display device 3 to the server 2 in order to update less current information. The communication between the communication device 21 and the server 2 may be executed by using any or all communication methodologies, including wired and wireless methodologies.

Further, as described above, images relating to a subject, or various information which may be relevant to a diagnosis to be performed by one or more medical workers may be received via communication with the server of the medical imaging system and/or the internet 14, another computer, or another medical device 3.

The input device 22 provides an interface which enables a user, such as, for example, a medical worker, who uses the medical device 3, to manipulate the medical device 3, and may include, for example, at least one of a remote controller, a mouse, a keyboard, a voice recognition device, a movement recognition device, and/or any other device which is suitable for receiving input from a user. The medical worker freely manipulates the medical device 3 by using inherent functions of respective devices, including input device 22.

When the display device 4 includes a touchscreen, functions of the medical device 3 may be manipulated by directly touching respective icons which are displayed on the display device 4. In particular, the display device 4 may function as the input device 22, in addition to performing the information display function.

The controller 23 controls operations of the medical device 3 based on information received via the communication device 21 and exterior commands received via the input device 22 and/or the display device 4. The controller 23 controls the display device 4 to display medical images, diagnosis records and the like, stores modified or updated medical images or diagnosis records, and transfers the stored information via the communication device 21 to the server 2 in order to store the same in the server 2.

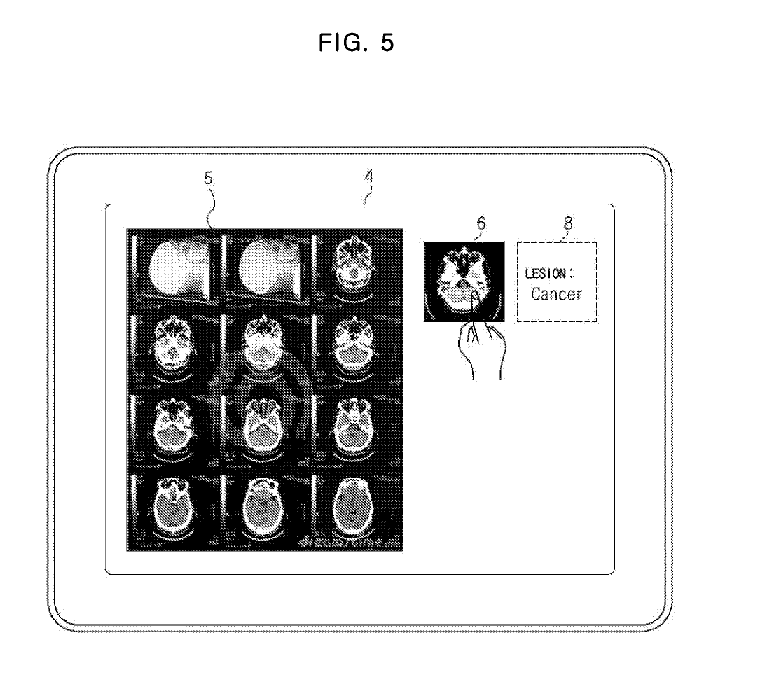

The display of medical images by the display device 4 will be described below with reference to FIGS. 5, 6, 7, 8, and 9 in more detail.

In an exemplary embodiment, when a doctor who first interprets medical images (hereinafter, referred to as “first doctor”) finds a lesion, interpretation records are entered via the medical device 3, and transferred to and stored in the server 2. When a particular medical image is a three-dimensional image, a corresponding amount of data relating to the particular medical image is great, and therefore, a considerable amount of time may be required in order to find lesions.

Conventional medical systems typically include various departments which are compartmentalized in order to efficiently and expertly treat diseases. The corresponding departments treat only diseases professionally handled thereby in order to provide specialized medical services to patients.

Accordingly, when abnormal unusual images which include at least one indication relating to a pathological entity, such as, for example, a lesion, are found as a result of interpretation of medical images by the first doctor, the patients are diagnosed and treated in detail in the department that specially treats the corresponding lesion.

After patient information and medical images are transferred to other departments, medical images are often redundantly interpreted anew by medical workers of the receiving department. When a particular medical image is transferred to the medical device 3 of the corresponding department and is displayed thereby, the diagnosis records made by the first doctor which relate to the particular medical image are accessible in conjunction with the particular medical image.

In particular, when the first doctor records abnormal unusual images and/or one or more lesions found in a patient as a result of interpretation of the images, subsequent medical workers see records relating to such lesions.

However, the subsequent medical workers see only opinions and records created by the first doctor which are associated with the one or more lesions, and it is thus necessary that an independent interpretation of the corresponding images is performed by at least one subsequent medical worker, similarly as performed by the first doctor, in order to check whether abnormal unusual states or lesions are present in three-dimensional images showing the body of the patient.

In a conventional hospital or health care facility, whenever records of particular patients are transferred to other departments or other medical workers, inefficiently, medical workers of the corresponding departments redundantly interpret three-dimensional images showing the body sites of the particular patients again.

Such an inefficiency may lead to a reduction in a number of patients that receive medical service, and consequently, a longer time is required for medical workers in order to perform diagnoses with respect to more patients. Thus, there is a demand for improvement.

Accordingly, in one exemplary embodiment, a plurality of images 5 relating to subjects are displayed on a medical device 3, and objects 6 and 7 which relate to a particular image 9 which includes an indication relating to a pathological entity, such as, for example, a lesion, from among the plurality of images 5 relating to the subject, are displayed by medical workers, to solve the aforementioned problems.

When the medical worker clicks or touches one or both of the objects 6 and 7 displayed by the display device 4 of the medical device 3, immediately, the image 9 which includes an indication relating to a pathological entity is displayed in accordance with the image interpretation result which is determined by one or more of the previous medical workers.

Accordingly, as described above, it is possible to eliminate an inefficiency relating to a redundant image interpretation which often occurs whenever records of patients are transferred to other departments or other medical workers.

Hereinafter, selected exemplary embodiments will be described with reference to the drawings in more detail.

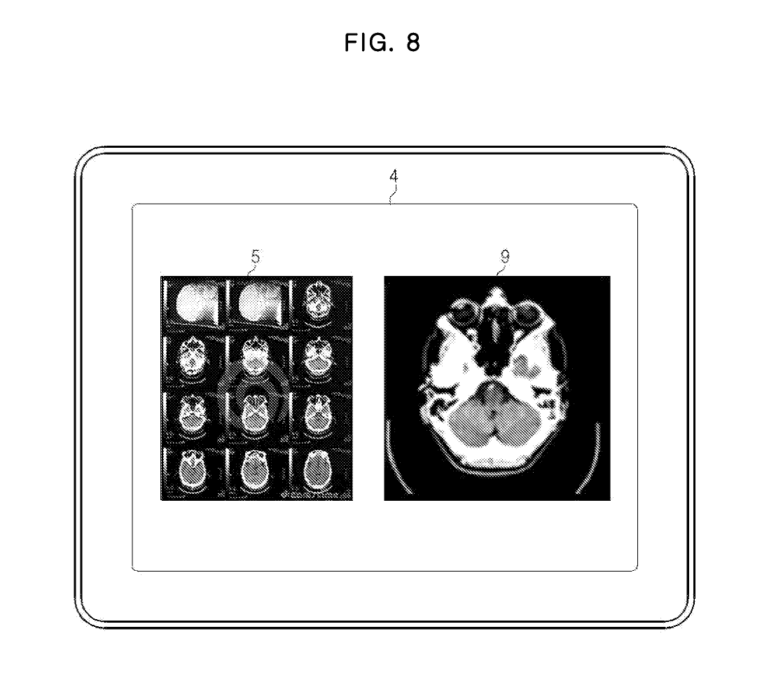

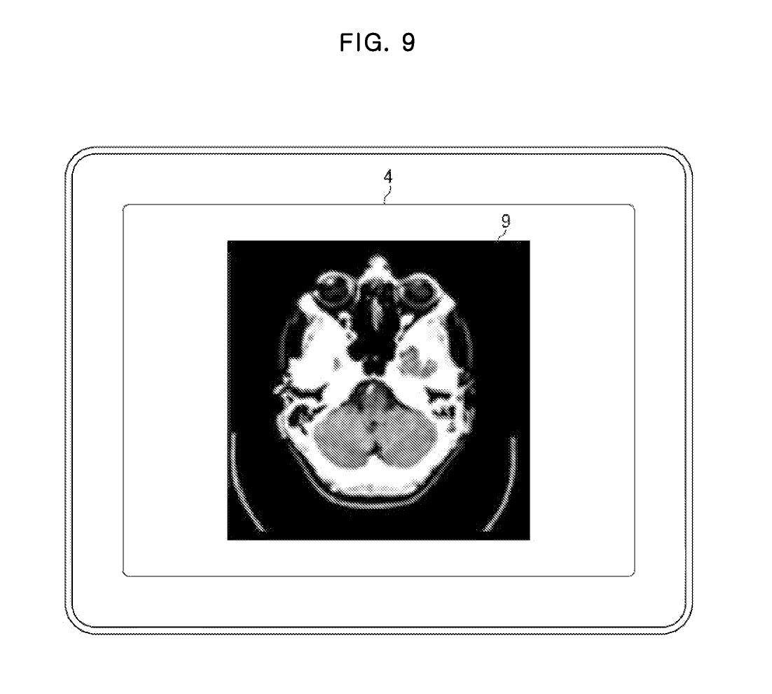

FIGS. 5, 6, and 7 are views which illustrate a process for displaying a medical image on a medical device 3 of a medical imaging system according to one exemplary embodiment, and FIGS. 8 and 9 are views which illustrate a state in which enlarged images which include respective indications relating to respective pathological entities are displayed on the medical device 3 of the medical imaging system according to one exemplary embodiment.

First, an image 5 relating to a subject, which image has been acquired by using an image acquisition device 1, is displayed on a display device 4 of a medical device 3.

The image 5 may be displayed as a three-dimensional image, or two-dimensional images constituting the three-dimensional image may be arrayed and displayed. A mode in which the image 5 is displayed may be selected by a user.

Further, the image 5 relating to the subject, and objects 6 and 7 which relate to the image 9 which includes an indication relating to a pathological entity, as determined by one or more medical workers, are displayed on the display device 4 of the medical device 3.

The image which includes an indication relating to a pathological entity may be an image that is considered to show a lesion therein or include an abnormal or unusual image, as a result of a corresponding image interpretation result which is determined by one or more medical workers. In particular, the term “lesion” refers to a pathological change of a human body, and includes diseases and other abnormalities.

The objects 6 and 7 may include a thumbnail 6 of the image 9 that is considered to have an indication relating to a lesion therein or to include an abnormal or unusual image, or a shortcut 7 to the image 9 that is considered to have an indication relating to a lesion therein or to include an abnormal or unusual image.

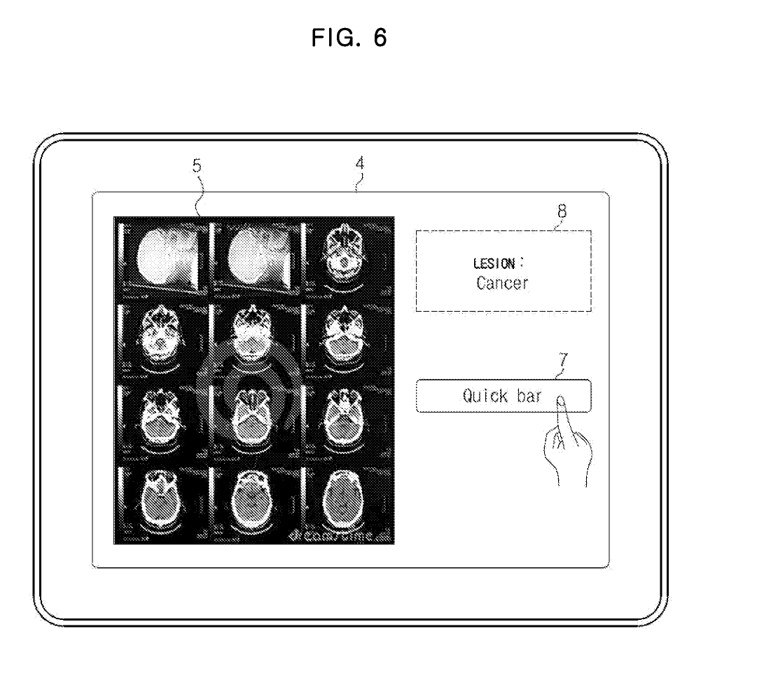

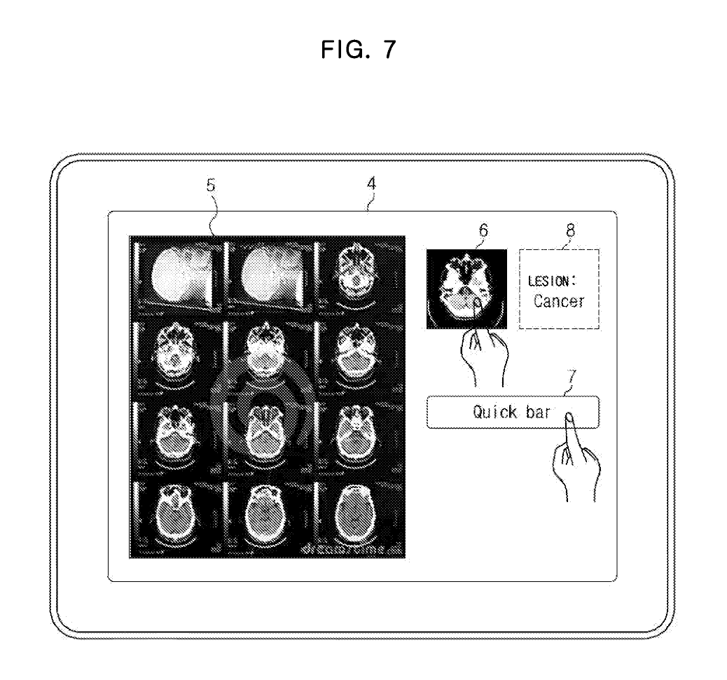

FIG. 5 is a view which illustrates a state in which a thumbnail 6 of the image 9 that is considered to have an indication relating to a pathological entity, such as a lesion, therein, or to include an abnormal or unusual image is displayed, as an example of the object, FIG. 6 is a view which illustrates a state in which a shortcut 7 to the image 9 that is considered to have the indication relating to the pathological entity therein or to include an abnormal or unusual image is displayed, as an example of the object, and FIG. 7 is a view which illustrates a state in which the thumbnail 6 is displayed in conjunction with the shortcut 7, as an example of the object. There is no limitation as to the format of the objects 6 and 7 which relate to the image 9 that is considered to have the indication relating to the pathological entity therein or to include an abnormal or unusual image. The objects 6 and 7 preferably include a thumbnail 6 which is easily objectively seen by a user.

When a user's command is provided with respect to at least one of objects 6 and 7 displayed on the medical device 3, the image 9 that is considered to have the indication relating to the pathological entity and which relates to the objects 6 and 7 is displayed in an enlarged form.

In particular, referring to FIGS. 5, 6, and 7, in a case in which the display device 4 of the medical device 3 adopts a touch screen, when medical workers touch at least one of the objects 6 and 7 displayed on the medical device 3, the image 9 that is considered to have the indication relating to the pathological entity and which relates to the objects 6 and 7 is displayed in an enlarged form on the display device 4, as illustrated in each of FIGS. 8 and 9.

Alternatively, when at least one of the objects 6 and 7 is clicked or selected by using an input device 22 which includes at least one of a remote controller, a mouse, a keyboard, a voice recognition device and a movement recognition device, the image 9 that is considered to have the indication relating to the pathological entity and which relates to the objects 6 and 7 is displayed in an enlarged form on the display device 4, as illustrated in each of FIGS. 8 and 9.

As shown in FIG. 8, the image 9 that is considered to include the indication relating to the pathological entity may be displayed together with the image 5 relating to the subject, such that the image 9 and the image 5 are divided on a screen of the display device 4, and as shown in FIG. 9, the image 9 may be displayed in an enlarged form on an entirety of the screen of the display device 4. A mode of display of the image 9 may be selected and varied by the user.

After a previous medical worker has generated records relating to a lesion, and selects and records an image which shows the lesion, when the next medical worker retrieves and displays the corresponding image on the medical device 3, the objects 6 and 7 which relate to the image 9 that is considered to include the indication relating to the lesion are displayed on the medical device 3, and the next medical worker immediately sees the image which shows the lesion by touching or clicking at least one of the objects 6 and 7, thereby precluding the inefficiency of redundantly interpreting or reviewing images anew in order to find the lesion.

Further, records 8 which relate to the lesion, which records have been generated by medical workers, are displayed on the display device 4 of the medical device 3. The records 8 relating to the lesion may include an opinion of one or more medical workers, possibly including names of diseases.

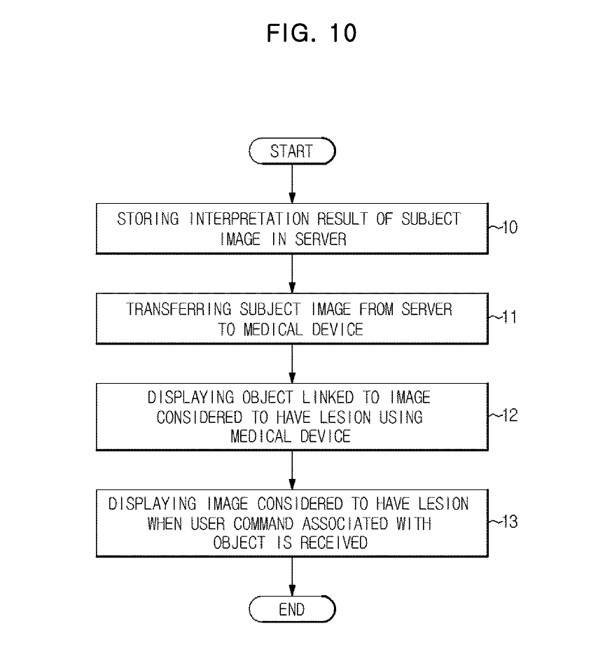

FIG. 10 is a flowchart which illustrates a method for displaying a medical image according to one exemplary embodiment.

Referring to FIG. 10, in operation 10, an interpretation result for an image relating to a subject is stored in a server 2.

When a medical worker interprets the image relating to the subject and records the interpretation result, that is, generates diagnosis records and the image 9 that is considered to have an indication relating to a pathological entity, such as a lesion, therein or to include an abnormal or unusual image, the information generated by the medical worker is transferred as data having a DICOM data format from the medical device 3 to the server 2 and stored in the server 2.

In operation 11, when the image relating to the subject is requested from the medical device 3, the server 2 transfers image information relating to the subject to the corresponding medical device 3.

The information transferred to the medical device 3 is transferred as data having a DICOM format, and the information includes the image relating to the subject and the interpretation results associated therewith.

In operation 12, when the image information relating to the subject is transferred from the server 2, the medical device 3 displays, on the display device 4, the image 5 relating to the subject, at least one of objects 6 and 7 which relate to the image 9 that is considered to have the indication relating to the pathological entity, such as a lesion, therein or to include an abnormal or unusual image, and records 8 relating to the lesion.

The image relating to the subject may be displayed as a three-dimensional image, or two-dimensional images constituting respective cross-sections of the three-dimensional image may be displayed in an array. The display mode may be selected by a user.

Further, the image 5 relating to the subject and the at least one of the objects 6 and 7 which relate to the image 9 which includes the indication relating to the pathological entity are displayed on the display device 4 of the medical device 3.

The objects 6 and 7 may include a thumbnail 6 of the image 9 which includes the indication relating to the pathological entity and/or a shortcut 7 to the image 9. One or both of the thumbnail 6 and the shortcut 7 are displayed on the display device 4. There is no limitation as to the forms of the objects 6 and 7 which relate to the image 9. The objects 6 and 7 preferably include a thumbnail 6.

Further, records 8 which relate to the lesion and which are generated by medical workers are displayed on the display device 4 of the medical device 3. The records 8 which relate to the lesion may include an opinion of one or more medical workers, possibly including names of diseases.

In operation 13, the medical device 3 displays the image 9 which includes the indication relating to the pathological entity and which relates to the objects 6 and 7, when a user command relating to at least one of the displayed objects 6 and 7 is received.

In a case in which the display device 4 of the medical device 3 includes a touch screen, when one or more medical workers touch at least one of objects 6 and 7 which is displayed on the medical device 3, the image 9 which includes the indication relating to the pathological entity and which relates to the objects 6 and 7 is displayed in an enlarged form on the display device 4.

Alternatively, when at least one of the objects 6 and 7 is clicked or selected by using an input device 22 which includes at least one of a remote controller, a mouse, a keyboard, a voice recognition device and a movement recognition device, the image 9 which includes the indication relating to the pathological entity and which relates to the objects 6 and 7 is displayed in an enlarged form on the display device 4.

After a previous medical worker has generated one or more records relating to a lesion, and selects and records an image which shows the lesion, when a subsequent medical worker retrieves and displays the corresponding image on the medical device 3, at least one of the objects 6 and 7 which relate to the image 9 which includes the lesion is displayed on the medical device 3, and the subsequent medical worker immediately sees the image 9 by touching or clicking the at least one of the objects 6 and 7, thereby avoiding the inefficiency of redundantly interpreting or reviewing images anew in order to find the lesion.

Although a few exemplary embodiments have been shown and described, it shall be appreciated by those skilled in the art that changes may be made in these exemplary embodiments without departing from the principles and spirit of the present disclosure, the scope of which is defined in the claims and their equivalents.

What is claimed is:

1. A medical device comprising:
a display device;
a communication device which communicates with an external device; and
a controller which displays an image which includes an indication relating to a pathological entity on the display device when the communication device receives image information relating to a subject.

2. The medical device according to claim 1, wherein the controller displays an object relating to the image which includes the indication relating to the pathological entity on the display device.

3. The medical device according to claim 2, wherein the object comprises at least one of a thumbnail and a shortcut of the image which includes the indication relating to the pathological entity.

4. The medical device according to claim 2, wherein the controller displays the image which includes the indication relating to the pathological entity in an enlarged form on the display device when a command relating to the object is received.

5. The medical device according to claim 4, wherein the display device includes a touch screen, and
the command relating to the object is received via a touch of the object displayed on the display device.

6. The medical device according to claim 4, wherein the medical device comprises an input device which includes at least one of a remote controller, a mouse, a keyboard, a voice recognition device and a movement recognition device, and
the command relating to the object is received via the input device.

7. The medical device according to claim 1, wherein the image which includes an indication relating to a pathological entity comprises an image which includes image information relating to a lesion.

8. The medical device according to claim 1, wherein the communication device communicates with at least one of a server, the internet and a second medical device in order to receive the image information relating to the subject.

9. A method for displaying a medical image comprising:
receiving a plurality of images relating to a subject; and
displaying a first image which includes an indication relating to a pathological entity from among the plurality of images relating to the subject on a medical display device, when the plurality of images relating to the subject are received.

10. The method according to claim 9, wherein the displaying the first image comprises:
displaying an object which relates to the first image on the medical display device; and
displaying the first image in an enlarged form on the medical display device when a command relating to the object is received.

11. The method according to claim 10, wherein the command relating to the object is received via a touch of the object displayed on the medical display device.

12. The method according to claim 10, wherein the command relating to the object is received via at least one of a remote controller, a mouse, a keyboard, a voice recognition device and a movement recognition device.

13. The method according to claim 10, wherein the object comprises at least one of a thumbnail and a shortcut of the first image.

14. The method according to claim 9, wherein the displaying the first image comprises:
displaying images relating to the subject on the medical display device and displaying at least one record relating to the first image on the medical display device.

15. The method according to claim 9, wherein the receiving the plurality of images relating to the subject includes receiving the plurality of images relating to the subject from at least one of a server, the internet and a medical device.

16. A medical imaging system comprising:
an image acquisition device which acquires a plurality of images relating to a subject; and
a medical display device which displays a first image which includes an indication relating to a pathological entity from among the plurality of images relating to the subject.

17. The medical imaging system according to claim 16, wherein the medical display device displays an object which relates to the first image.

18. The medical imaging system according to claim 17, wherein the object comprises at least one of a thumbnail and a shortcut of the first image.

19. The medical imaging system according to claim 17, wherein the medical display device displays the first image in an enlarged form on the medical display device when a command relating to the object is received.

20. The medical imaging system according to claim 19, wherein the command relating to the object is received via a touch of the object displayed on the medical display device.

21. The medical imaging system according to claim 19, wherein the command relating to the object is received via at least one of a remote controller, a mouse, a keyboard, a voice recognition device and a movement recognition device.

22. The medical imaging system according to claim 16, wherein the medical display device includes at least one of a computer, a smart phone and a tablet personal computer (PC).

23. The medical imaging system according to claim 16, wherein the medical display device displays at least one record relating to the first image.

24. The medical imaging system according to claim 16, wherein the first image comprises an image which includes image information relating to a lesion.

25. The medical imaging system according to claim 16, further comprising:
a server which stores the acquired plurality of images relating to the subject and which transfers the stored plurality of images to the medical display device,
wherein the server supports a digital imaging and communication in medicine (DICOM) protocol.

26. The medical imaging system according to claim 16, wherein the image acquisition device acquires three-dimensional images relating to the subject.

27. The medical imaging system according to claim 16, wherein each of the image acquisition device and the medical display device supports a DICOM protocol.
